(19) United States
(12) Patent Application Publication
(10) Pub. No.: US 2020/0297339 A1
(43) Pub. Date: Sep. 24, 2020

(54) INTERVENTIONAL RADIOLOGY SUTURE DEVICE

(71) Applicant: DARCY SHAW, LEAWOOD, KS (US)

(72) Inventor: DARCY SHAW, LEAWOOD, KS (US)

(21) Appl. No.: 16/827,297

(22) Filed: Mar. 23, 2020

Related U.S. Application Data
(60) Provisional application No. 62/821,490, filed on Mar. 21, 2019.

(51) Int. Cl. A61B 17/04; A61B 34/20

(52) U.S. Cl. CPC A61B 17/0469; A61B 2090/3966; A61B 34/20

(57) ABSTRACT

The present invention is directed towards an interventional radiology suture device which comprises a head configured for slidable receipt of a plunger assembly, a shaped cannula extending outwardly from the head for transmitting a threaded suture, a central body extending between the head and the shaped cannula, the central body presenting a channel for receiving a cartridge of threaded sutures which are dispensed though the shaped cannula towards a surgical site.

### FIELD OF THE INVENTION

The present invention is broadly directed to a repair tool for use during interventional radiology and more particular to an interventional radiology suture device which delivers sutures to a surgical site and allows for fixating the sutures using appropriate tension at the surgical site which can be viewed with traditional radiology equipment. For example, soft tissue herniations caused by defects, abnormalities or injuries typically located within a patient's abdominal cavity may be sutured using the interventional radiology repair tool.

### BACKGROUND OF THE INVENTION

As medical and hospital costs continue to increase, surgeons are constantly striving to develop advanced surgical techniques. Advances in the surgical field are often related to the development of operative techniques which involve less invasive surgical procedures and reduce overall patient trauma. In this manner, the length of hospital stays can be significantly reduced, and therefore the hospital and medical costs can be reduced as well. Conventional sutures typically utilize a knot for placement of a stitch to ensure proper healing often times these knots are hand tied into the threaded member with a second knot tied abound the tissue to be joined. However, the formation of the knot on the threaded member is tedious and time-consuming during the surgical procedure within a tightly-confined space.

Suturing is used in many surgical operations to close, i.e. bring together, tissue sections which have been separated, e.g. by cutting. For example, during hernia repair or c-section surgical operations, the abdominal fascia tissue is surgically cut, and then closed by stitching it back up (or sutured together). Sutures are used in order to fix and maintain the cut tissue adjacent to each other during the healing period. A number of post-surgical complications can be caused by uneven or improper sutures, tension or tearing of the suture thread from the engaged tissue, creating significant risks or complications to patients. Such post-surgical complications may also be of concern to care-takers and medical insurance organizations.

One common repair is the results from herniated tissue. Annually, there are over 800,000 inguinal hernia repairs along with an additional 400,000 ventral repairs required. If repaired, the recidivism rate of future hernias is high with 63% likely to reoccur within ten years from an incisional repair alone and 32% are likely to reoccur if prosthetic mesh is utilized in the repair.

Traditional suturing involves using a needle threaded with a suitable suturing thread, that is operated by the surgeon by hand or by using forceps. The surgical site is opened up by an incision over the defect so that the surgeon has a direct view of the hernia or site to be repaired. The incision is retained in an open position so that the muscle tissue surrounding the defect can be joined together to repair the defect by passing a needle through surrounding tissue sections, and the thread is pulled tight to bring the surrounding tissue sections closer together. The incision is then closed with stitches, staples, surgical tape or special glue. Often traditional repair procedures have a number of risks, including increased risk of reinjury, excessive bleeding, infection, numbness, pain, becoming chronic, anesthesia risks, mesh complications, damage to patent organs or organ functions.

Additionally, as a result of surgery, the chance of an adverse effect on the healing of abdominal incisional wounds can increase and may lead to the formation of tissue adhesion which may contribute to delayed healing, bowel obstructions, recurrent hernias or chronic wounds. As a result of the increased, treatment of small hernias is less common with treatment typically being offered only once the hernias become large and symptomatic. Over time, the chances that the wound will heal become less favorable. With morbid obesity on the rise, in some cases, the patient is required to lose weight prior to surgery. In emergency cases, the obesity of the patient must be weighted against the severity of tissue injury and, in some cases, may not be recommended. Historically, the adverse effects of surgery increase based on the patient's obesity increasing the risk of adverse surgical outcomes, incision size and necessary healing periods along with chances of recidivism.

During traditional fascia suturing procedure, a single, continuous thread is used and the needle is consecutively passed through both tissue sections in order to obtain a single, continuous suture. The suture is secured at both ends by a knot. Such traditional suturing procedures typically requires the involvement of at multiple surgical staff members, one member maintaining the tissue sections to be sutured in proper orientation with respect to one another, and the other staff member for performing the suturing procedure. As a single thread is used, there is a risk of the suture tearing during the procedure. In addition, due to the thickness of the fascia tissue layer and the relatively high force required to pass the needle through it, surgeons often suffer from needle pricks during surgery that may pose a health risk and exposure to contaminations to both the surgeon and the patient. Further, due to uneven tensioning or slaking of the thread, ischemic damage in vicinity to the suture may be formed.

Some alternative devices and suturing methods have been proposed in the art. One of the common techniques used is stapling, in which the tissue sections are held in proximity to one another by metal staples. Although stapling is a procedure that is quicker than traditional suturing, it is unsuitable for holding together thick tissues and is, thus, used in cosmetic or superficial skin incisions (i.e. incisions that are not carried out in the deep-tissue). Another proposal is to use mesh or a series of clips such as tacks, staples or sutures in rapid succession. Installing mesh may involve performing a laparoscopic procedure by going into the abdomen with a surgical tacking device and delivering a series of clips. This may increase the healing period, pain and complications associated with surgical procedure. Additionally, unfurling the mesh and attaching it to the fascia may fail or increase the healing period, pain and complications including potential bowel obstructions or adhesions.

Recent advances in medicine have led to a reduction in the invasiveness of surgical procedures through endoscopic surgery. Endoscopic surgery involves the use of an endoscope, which is an instrument permitting the visual inspection and magnification of any cavity of the body. In some of these procedures, the endoscope is inserted through a cannula, after puncturing a hole into the soft tissue protecting the body cavity. The hole is made with a trocar, which is a sharp-pointed instrument, often inserted within a trocar cannula. The obturator will puncture at least one hole into the tissue and in some cases, many holes are required until the correct size is obtained. When the necessary size opening is finally obtained, the obturator is removed from the trocar cannula and the surgeon can begin working on the necessary procedure. Often the procedure involves both diagnostic and therapeutic procedures at the surgical site with the aid of specialized instrumentation designed to fit into the opening created with the trocar cannula and any additional trocar cannulas required to provide the desired openings into the body cavity.

Because surgeries with large incisions have the likelihood to cause larger issues, minimally invasive surgery has advanced based in part to the use of medical tools and equipment along with medical imaging equipment which allows the surgeon to view the surgery from afar. Many of these minimally invasive surgeries involve laparoscopy, thoracoscopy, arthroscopy, intraluminal endoscopy, endovascular techniques, catheter-based cardiac techniques (such as, by way of example, and not limitation, balloon angioplasty), and interventional radiology. They are further beneficial because they can decrease the pain and healing time typically required after surgery with larger incisions. In some cases, sutures and ligatures have been replaced with various staples, clips or fasteners for securing ends of a defect or wound. As vascular surgery has transitioned to endovascular surgery and interventional radiology, such fasteners do not work or adequately replace traditional sutures. For example, when a cylindrical graft is inserted into an abdominal cavity during an open surgical procedure, ends of the graft may be secured to the ends of the retained vasculature using sutures. During endovascular repair of abdominal aneurysms, a stent may be used to secure the graft. However, stents may fail, resulting in a phenomenon known as a “Type I endoleak,” i.e., where the blood leaks around the stent-graft into the aneurysm cavity. This type of leak may lead to rupture of the aneurysm, re-operation, and/or increased risk of death of the patient.

The interventional radiological approach may be beneficial for treating minimally symptomatic hernias where the cost benefit analysis of the risk of adverse effects may be reduced in comparison to the size of the incision and concomitant healing period allowing for a shift in the benefit analysis. By reducing the incision, repair of the defective tissue may be treated without mesh. In addition, by utilizing a minimally invasive procedure like interventional radiology, an obese patient may be treated without the necessary delay for weight reduction and they may be able to avoid the other potential risks like those typically associated with anesthesia or abdomen wall incisions.

However, when interventional radiology is utilized, visibility is challenged and it is more difficult to preform a minimally invasive repair of defective tissue without direct visibility of the torn tissue. Often times, specialized equipment must be used to determine the exact location in three-dimensional space of the surgical instruments. In some cases, x-ray guided equipment is used to visualize the interior of the surgical site. One example is the use of a fluoroscope (a 30 Hz X-ray machine) which uses a continuous X-ray beam with a special machine called a C-arm. The C-arm can be used to rotate around a person's body to create a sequence of images that are projected onto a fluorescent screen, or television-like monitor. The fluoroscope allows a physician to view internal organs and tissues in motion. Still images are also captured and stored electronically on a computer with a camera tool. In some cases, during a fluoroscope procedure, images are taken of the subject tissue at a rate of 30 images per second. The images are then displayed on the TV-like monitor. In order to see the local structure of the internal area more clearly, some physicians inject a contrast material like a radio opaque dye into the patient. As the dye flows into the patient's bloodstream, it temporarily renders the injected area opaque, creating a silhouette on the fluoroscope monitor. Depending on the amount of dye introduced, the rate at which it is introduced, and the blood flow rate, the opacification lasts from 2-5 seconds. As new blood arrives, the dye is diluted and swept away and the opacification gradually fades. This procedure can be very uncomfortable for the patient since the dye causes a burning sensation as it enters the bloodstream.

During a typical image guided radiology intervention, the patient is continuously monitored by means of X-rays. The C-arm X-ray apparatus may be used to determine the position of a surgical instrument by carrying out X-ray fluoroscopy in two different, substantially perpendicular directions. The two images are simultaneously available to the surgeon, so that a three-dimensional impression can be generated by a computer to provide position and orientation data in the region of the surgical site. During the surgery, it is often necessary to monitor the operation by repeating the fluoroscopy of the patient for short time intervals so as to track the evolution of the surgery its progression. The procedure is slow, complex and time-consuming and, may create an excessive exposure to ionizing radiation both for the patient as well as the surgeon. In addition, in some cases, surgical instruments especially the tips appear to be transparent and are not clearly visible. Thus, in some cases, it is difficult to determine the position and orientation of the surgical instrument during the surgical procedure.

Therefore, there is a need for an improved interventional radiology hernia repair tool which addresses at least some of the problems and limitations associated with the aforementioned shortcomings and which can be used for delivering a fixation device and for securing the fixation device along plural locations associated with a surgical site within an internal cavity such as one associated with a tissue defect, such as but not limited to, a hernia.

### SUMMARY OF THE INVENTION

The present invention generally includes a suturing device and a system having an endoscopic shaped cannula for passage of an anchoring device to a surgical site. Generally, the endoscopic shaped cannula has an edge and a tip which is visible through an interventional radiology camera. The suturing device is generally adapted for tissue approximation by inserting a first anchoring structure connected on one end to a threaded member. The second end of the threaded member is connected to a second anchoring structure and tension is selectively applied to reduce undesired slack from the threaded member. Generally, the threaded member has a predetermined length.

The present invention also provides an interventional radiology suture device which comprises a head configured for slidable receipt of a plunger assembly; a shaped cannula extending outwardly from said head for transmitting a threaded suture; a central body extending between said head and said shaped cannula, said central body presenting a channel for receiving a cartridge of threaded sutures; said cartridge being in operable communication with said plunger assembly for dispensing at least one of said threaded sutures through said shaped cannula.

The shaped cannula extends from a proximate end to a distal end and has a cross-section includes at least one shaped edge adapted for at least partially separating surrounding fascia, thoracic wall or thick muscular tissue, etc. while moving the tip to the first side or the second side. The shaped cannula extending between the proximate and distal tip, the distal tip being adapted for rotation from the first side to the second side.

The endoscope includes a central passage which has a first opening associated with a plunger and a second opening associated with the shaped cannula. The central passage presents an opening adapted for receipt of a suture cartridge. Generally, the cartridge includes a plurality of anchoring structures joined together by the threaded member, the anchoring structures positioned for intersection by a plunger rod which extends through the central passage and extends the anchoring structures along with the threaded member which is secured by the anchoring structure to the first side.

After anchoring of the threaded member to the second side, the shaped edge (or another shaped surface) may be used to separate the threaded member. Each end of the threaded member includes an anchor which may be used for anchoring the threaded member at spaced apart anchoring positions. Generally, the anchor may include a shaped tip for anchoring the threaded member.

These and various other advantages and features of novelty which characterize the present invention are pointed out with particularity in the claims annexed hereto and forming a part hereof. However, for a better understanding of the invention, its advantages and objects obtained by its use, reference should be made to the drawings which form a further part hereof, and to the accompanying descriptive matter, in which there is illustrated and described preferred embodiments of the present invention.

### BRIEF DESCRIPTION OF THE DRAWINGS

FIGS. 1-7 illustrate a front perspective of an exemplary embodiment of the interventional radiology suture device used to suture a first tissue section and a second tissue section while closing an intervening space.

FIG. 8 is a side elevation of an exemplary embodiment of the interventional radiology suture device.

FIG. 9 is a front cross-section of an exemplary embodiment of the interventional radiology suture device taken along Line 9-9 in FIG. 8.

FIG. 10 is a front cross-section of an exemplary embodiment of a suture assembly extending from an exemplary plunger rod.

FIG. 11 is a top plan view of the embodiment of the interventional radiology suture device depicted in FIG. 8.

FIG. 12 is a bottom plan view of the embodiment of the interventional radiology suture device depicted in FIG. 8.

FIG. 13 is a front cross section of an alternative embodiment of the interventional radiology suture device with a second cannula.

FIG. 14 is a top plan view of the alternative embodiment of the interventional radiology suture device depicted in FIG. 13.

FIG. 15 is a bottom plan view of the alternative embodiment of the interventional radiology suture device depicted in FIG. 13.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 10 generally refers to an embodiment of the present invention, an interventional radiological suture device (also referred to as the “IR suture device” or simply the “device”) according to the present invention for laparoscopic, open procedures or generally where adjacent tissue segments separated by a defect, such as, but not limited to, a hernia are approximated. The IR suture device 10 includes a head 11 separated from a shaped cannula 12 by a central body 13. In the embodiment depicted in FIG. 12, the shaped cannula 12 generally has an elliptical-shaped, cross-section although other shapes may be utilized which achieve the same functionality as further described below. The shaped cannula 12 provides the functionality of providing a passage for the transport of a pair of threaded sutures 30, also referred to herein as anchoring structures to the surgical site. The shaped cannula 12 functions as a surgical tool which manipulates the relevant tissue and also provides the functionality of receiving and dispensing the pre-threaded sutures 30 to the desired surgical site.

The shaped cannula 12 also allows functions as an injection lumen for the injection of a saline or a contrasting fluid such as a dye or other type of contrast enhancing mediums which can be introduced through the shaped cannula at any point in the procedure if desired. The contrasting fluid can be injected to the site within the current shaped cannula 12 or a second cannula may be provided which runs parallel to the shaped cannula 12 for injection of the fluid with a syringe or other fluid delivery. In this way, the IR suture device 10 provides contrasting fluid at a uniform rate near the desired site for improved visualization and repair of the tissue defect.

In one embodiment, each threaded suture 30 includes at least a first anchor 30a joined to a second 30b by a threaded member 40. As desired, the threaded suture 30 may include additional anchors which are joined by the threaded member 40. Generally, the threaded member 40 may be a single or multiple stranded material by using various types of filaments of wire, silk, cotton, Dacron, etc. . . . The plurality of threaded sutures 30 are generally housed within a cartridge 32 and dispensed through the shaped cannula 12. The cartridge 32 is generally configured for housing and dispensing plural interconnected anchoring structures 30 utilizing at least one anchor 30a secured to one end of the threaded member 40.

In addition, the IR suture device 10 provides a combined approach to quickly repairing damaged tissue by quickly deploying a threaded suture 30 at the damaged site for fixation of the tissue along with any mesh material which can also utilize radiologic guidance in a percutaneous manner.

Generally, the shaped cannula 12 extends outwardly from the head 11 for dispensing the threaded suture 30 from the cartridge 32 to the surgical site. The shaped cannula 12 generally includes an inner lumen for transmission of the threaded suture 30 having sufficient size and dimensions for generally unimpeded passage of the threaded suture 30. In addition, the outer surface of the cannula 12, being adapted for manipulation of the surrounding tissue, includes a longitudinally extending edge 14 for manipulating the tissue. As is generally known, the edge 14 may be pointed, round or dull as needed for manipulation of the surrounding tissue. In addition, the cannula 12 can optionally include a plurality of indicia 38 spaced regularly along the outer surface according to the desired measuring needs. Each of the plural indicia 38 may, in one embodiment, refer to a distance and are spaced along the surface of the outer cannula 12.

A tip 16 extends from the end of the cannula 12 opposite the head 11. The tip 16 extends circumferentially along the cannula 12 with a centrally aligned aperture for passage of the threaded suture 30 to the surgical site. In addition, the tip 16 includes a radiological opaque material or coating which provides visibility of the tip 16 through an interventional radiology camera during for example, a surgical procedure. A radiopaque coating may be used to at least partially coat the surface of the tip 16, each indicia 38 and each anchor 30a, 30b to allow them to be viewed by fluoroscopy imaging systems to indicate position of the cannula 12 in reference to the defect 2, for example.

The radiopaque coating may include materials which are suitable for use as coatings in medical procedures which are non-toxic and have limited adverse effects to a patient and may include, but are not limited to, iodine, barium-sulphate, gadolinium, various phosphates, sulfates or heavy metal salts which becomes visible when struck by a high-energy beam of electrons. Coatings in accordance with the invention typically contain 10 wt % or more of one or more radiopaque materials (e.g. from 10 wt % to 25 wt % to 40 wt % to 50 wt % to 60 wt % to 70 wt % to 80 wt % to 90 wt % to 95 wt % to 97 wt % to 99 wt % or more). The tip 16, being radiologically opaque, allows for the location of the threaded suture 30 to be depicted through the interventional radiological device. In this way, the visibility of the deployment of the threaded suture 30 through the IR suture device 10 is enhanced.

As further illustrated in FIG. 9, the IR suture device 10 includes a central body 13 with a central passage 24 which extends between an outer body 18 and the cannula 12. The central passage has sufficient dimensions for receipt of the cartridge 32 with plural threaded sutures 30. The cartridge 32 is configured for housing and distributing the threaded sutures 30. In addition, the cartridge 32 has complementary structure for delivery of the threaded sutures 30 through the cannula 12.

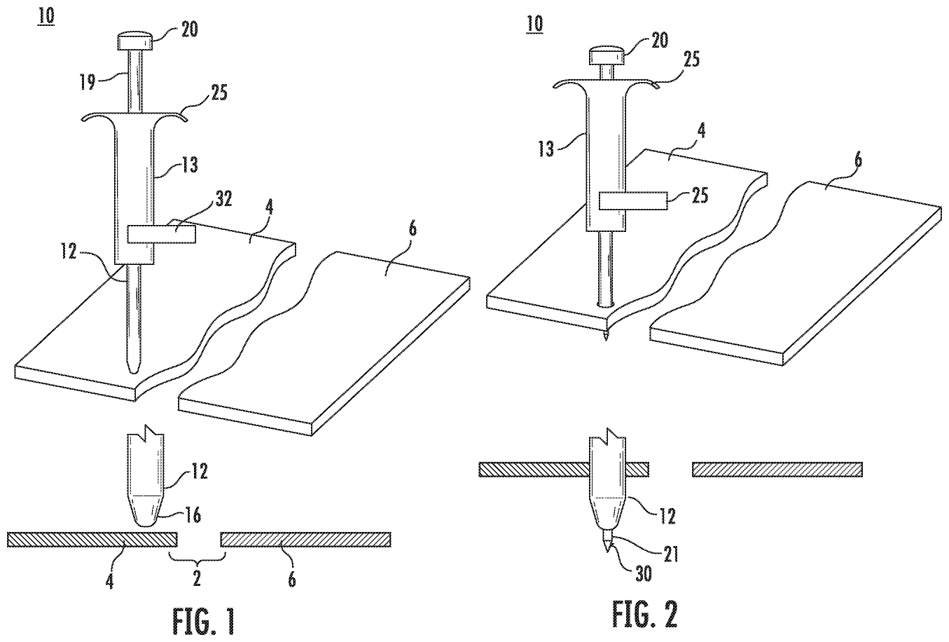

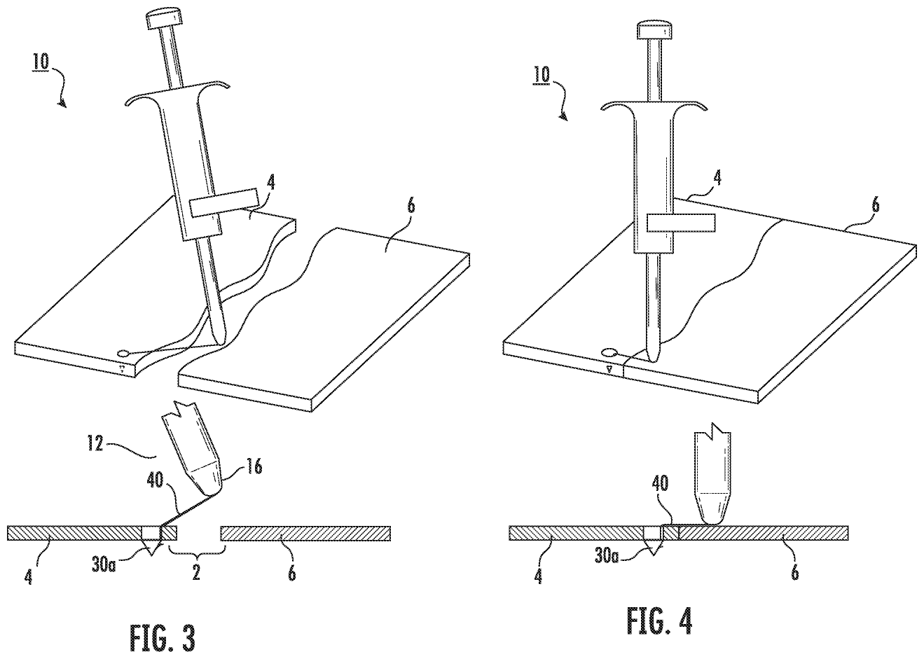

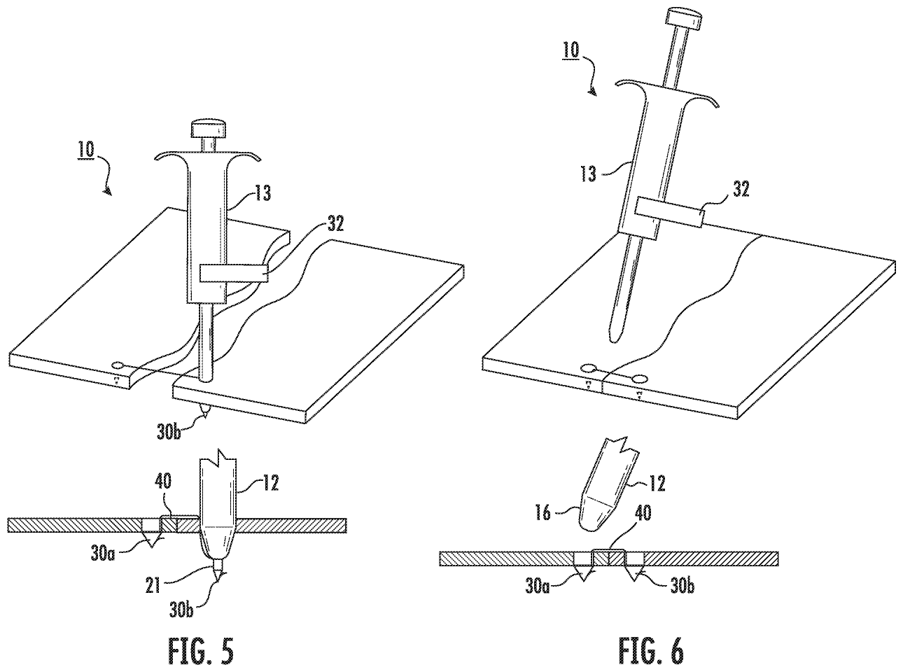

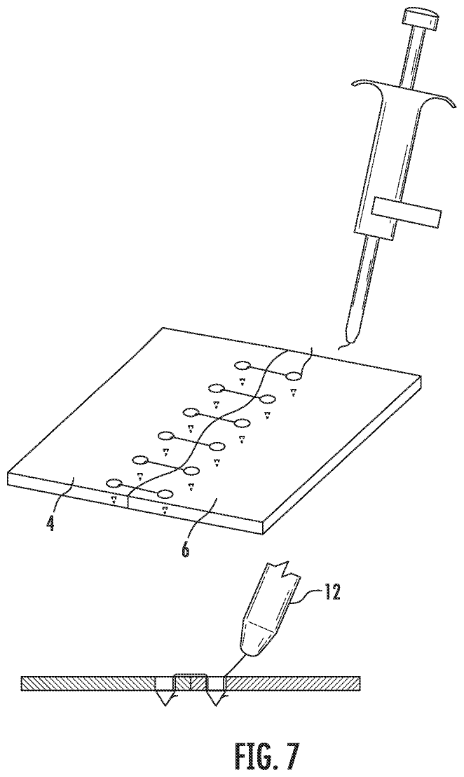

Cartridge 32 is configured for receiving a variety of threaded sutures 30 with different sized anchors 30a, each having a different dimension, size, diameter or length and for a variety of different threaded members. The cartridge 32 is depicted in FIGS. 1-7, 9, 13 as being rectangular for receipt within the channel 36, which is also depicted as being generally rectangular. The channel 36 is located within the central body 13 and configured for slidable receipt of the cartridge 32. In addition, the channel 36 may include additional mechanical structure for securing the cartridge 32 during receipt by the IR suture device 10 for dispensing the threaded sutures 30 through the shaped cannula 12 to the surgical site.

The cartridge 32 is shaped for receipt within the central body 13 for mechanical engagement by a plunger assembly 26 for deploying at least one of the interconnected anchoring structures 30 towards the defect 2 for joining the surrounding tissue 4, 6. The plunger assembly 26 generally extends between the outer body 18 and the shaped cannula 12.

The suture cartridge 32 provides for use of the IR suture device 10 with a variety sized interconnected anchoring structures 30, each with at least one anchor 30a and an interconnected threaded member 40 for securing the anchor and joining the surrounding tissue. Generally, the suture cartridge 32 is adapted for removeable receipt by the channel 36, the suture cartridge 32 having plural interconnected anchoring structures 30, each joined with the threaded member 40 have a variety of possible sizes depending on the desired property of the anchors 30a or threaded member 40. Generally, the suture cartridge 32 allows for placement of interconnected anchoring structures 30 for intersection by a plunger rod 21 which extends through the central passage and extends the individual anchors 30a along with the threaded member 40 which is secured to the first side 4.

The interconnected anchoring structure 30 is adapted for one-way insertion into tissue surrounding a tissue defect and closing the defect without the need for a surgical knot while maintaining a flexible connection between the pair of anchors 30a with the threaded member 40. In addition, the interconnected anchoring structure 30 can be stored within a cartridge 32 containing a plurality of anchoring structures 30 which can be mechanically transmitted down the cannula 12 from the central body 13 with the plunger assembly 26.

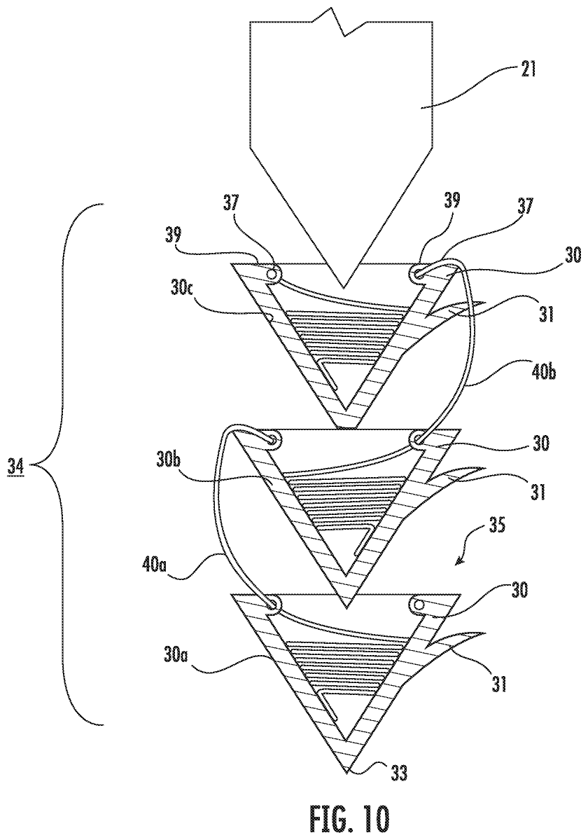

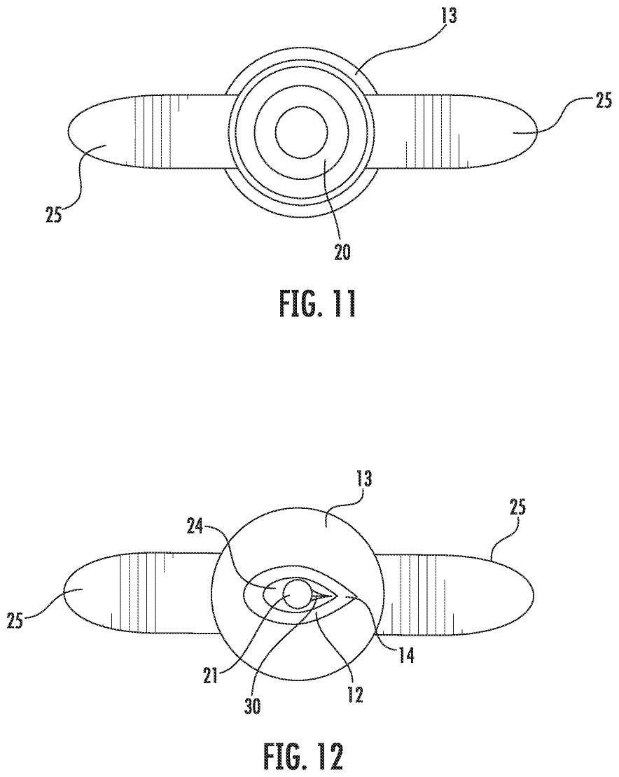

An embodiment of the suture assembly 34 is depicted in FIG. 10. In the depicted embodiment, the anchor 30a is generally conically shaped for receipt of the plunder assembly 26, the anchor 30a extending from an open end 35 tapering towards a tip 33 which is adapted for insertion through tissue. The anchor 30a is generally secured from undesired tissue removal with an embodiment of a barb 31, illustrated near the open end 35.

The threaded member 40 is secured to the inner projection 39 of the first anchor 30a and the second anchor 30b. An example of an embodiment of the inner projection 39 is illustrated in FIGS. 9-10 as an eyelet 37, positioned near the open end 35 and extending inwardly from the anchor 30a. The exemplary embodiment of the eyelet 37 secures each end of the threaded member 40 to the anchor 30 supplying the desired tension for suturing the surrounding tissue.

The anchor 30a is generally adapted for transmission along the cannula 12 by the plunger rod 21 and for adjustable receipt of the threaded member 40. In the embodiment depicted in FIG. 10, the plunger rod 21 has complementary structure for receiving and transmitting the suture assembly 34 along the cannula 12.

In operation, the interconnected anchoring structures 30 are housed within the cartridge 32. The cartridge 32 is slidably secured to the channel 36 for deployment by the plunger assembly 26 through the shaped cannula 12. Upon deployment of the first anchor 30a from the shaped cannula 12 by the plunger assembly 26 through one of the first or second tissues 4, 6, the tip 16 is directed to the opposite tissue using visual and radiologic confirmation of the tissue layers. To accomplish this, the contrasting medium may be injected to the correct layer under radiologic guidance. After securing the first anchor 30a to the first tissue 4, the shaped edge 14 can be brought towards the second tissue 6 by guiding the shaped edge 14 to the point of palpable tension. The plunger assembly 26 may be utilized to deploy the second anchor 30b and with radiological guidance can be secured to the second tissue 6 to approximate the tissues 4, 6 securely. Once the second anchor 300b is deployed, the shaped cannula 12 can be removed and another cartridge 32 utilized to provide an additional suture. By way of example, the next cartridge 32 may include a threaded member 40 which is shorter than the previous cartridge 32 to facilitate tighter tissue approximation, closing the tissue defect.

The barb 31 is designed to allow travel in one direction such as passage through the central passage or lumen 24 and into the tissue surrounding the defect 2 while the barb 31 limits travel in the opposite direction so that removal of the anchor 30a from the tissue is difficult. The threaded member 40 associated with the interconnected anchoring structure 30 extends between a pair of barbed anchors 30a also referred to herein as anchoring structures or anchors which secure the threaded member 40 to the tissue.

The threaded member 40 depicted in FIG. 10, is connected between the barbed anchors 30 and when the anchor 30 is inserted through a first-tissue section 4 it may be used to close a defect between adjacent first and second tissue sections 4, 6. Threaded member 40 may include traditional suture materials which are used for sewing or tying tissues and may include absorbable material such as, but not limited to, biological tissue, synthetic fibers including polyesters, co-polymers and a blend of and non-absorbable materials like, but not limited to silk, cotton or nylon, which are generally non-irritant and which may disintegrate or become digested after a period of time. The threaded member 40 may also be monofilament or multifilament in a braided, spun or twisted arrangement. The threaded member 40 will also have a diameter based upon the desired strength properties. Typically, the diameter and length of the threaded member 40 will vary dependent on the location of the injury, desired strength and healing period. The length of the threaded member 40 will also vary as needed.

In the embodiment depicted in FIG. 9, the outer cylindrical body 18 associated with the head 11 of the central body 13 receives the plunger assembly 26. Generally, the plunger assembly 26 includes the outer cylindrical body 18 in slidable receipt of a plunger 19 with a top 20 and seal 23, an optional spring 27 positioned therebetween. The seal 23 is depicted as a generally conventional sealing membrane which encircles the plunger 19 and is spaced opposite the top 20. The seal 23 generally seals the plunger assembly 26 to limit escaping fluid or air from the outer cylindrical body 18, which when the plunger 19 moves down the outer cylindrical body 18, it compresses the air and provides for pneumatic operation of the interconnected anchoring structure 30 during movement of the anchor 30a.

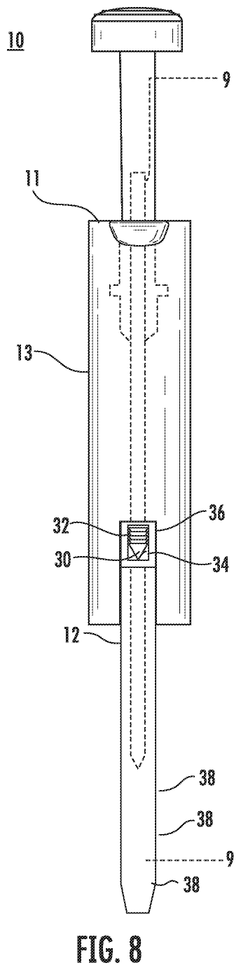

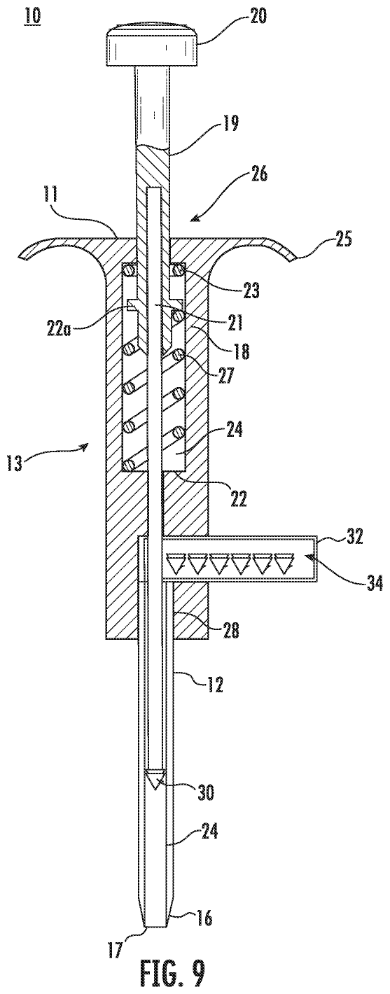

In operation, the plunger 19 is slid down the outer cylindrical body 18 as the top 20 is depressed, the seal 23 being generally positioned therebetween. The top 20 has a central surface for engagement by a thumb or finger, for example, compressing the air and generating the pneumatic pressure to assist in movement of the interconnected anchoring structure 30 from the cartridge 32 along the cannula 12 to the area of the tissue defect 2. In the embodiment depicted in FIGS. 9-8, a plurality of flutes 25 extend from the outer cylindrical body 18. The flutes 25 can assist with the grip of the IR suture device 10 during use and allow for greater control and operation of the plunger assembly 26 as the interconnected anchoring structure 30 is deployed to the defect 2 through the cannula 12 for suturing the surrounding tissue.

As indicated above, in one embodiment of the plunger assembly 26 may include a spring-like biasing member, also referred to as the spring 27. The spring 27 applies a reactionary force to the underside of the top 20 during depression of the plunger 19 as the plunger 19 moves from an upper position to a lower position, helping the plunger 19 return towards the upper position where the spring 27 is uncompressed. Generally, the spring 27 is positioned between the outer cylindrical body 18 and the plunger 19 for engagement by the top 20 and an internal support wall 22 as the plunger 19 is depressed.

Alternatively, the plunger 19 may include a circumferential ring 22a extending radially from the plunger 19, the circumferential ring 22a having a diameter greater than the plunger 19, for retaining the spring 27 within the central body 13 during operation. Generally, the circumferential ring 22a is positioned between the seal 23 and the internal support wall 22 along the plunger 19.

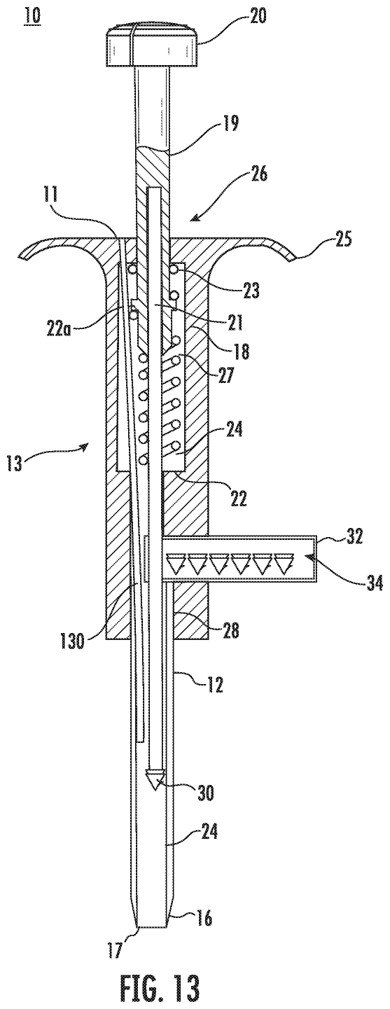

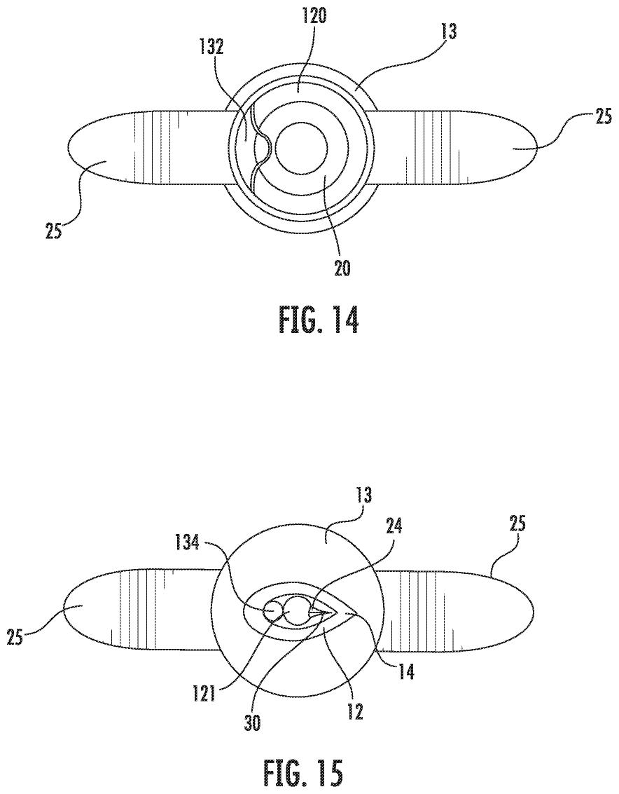

A second embodiment of the interventional radiological device is illustrated in FIGS. 13-15 with a second cannula 130 extending through the central body 13 and presenting a second channel which extends into the shaped cannula 12. The second cannula 130 extends between a secondary top 130 and a secondary passage 134. The secondary top 130 is provides for independent operation from the primary top 120 and is generally ergonomic and complementary shaped in relation to the primary top 120. Generally, the second cannula 130 is configured for receipt of a syringe needle for receipt of a contrasting fluid which can be injected into the surgical site to enhance the visibility of the site during use of interventional radiological equipment. The second cannula 130 provides the function of transmitting a contrasting fluid independently from the plunger assembly 26 to the surgical site. The second cannula 130 depicted in FIG. 13 extends along the plunger assembly 26 running into the shaped cannula 12. Alternatively, second cannula 130 can be spaced from or integrated into the plunger assembly 26 while providing independent access for transmission of the contrasting fluid. This may be done, by way of example, by positioning it centrally within the shaped cannula 12.

As illustrated in FIGS. 1-7, in operation, the suturing device 10 is adapted for suturing at least one tissue section to another by inserting the interconnected anchoring structure 30, with a first and second anchor 30a, 30b interconnecting the threaded member 40 between the first (tissue) side 4 and the second (tissue) side 6. As the plunger 19 is depressed, the plunger rod 21 intersects the suture assembly 34 and deploys the first anchor 30a with connected threaded member 40 along the shaped cannula 12 for insertion into the tissue near the defect 2. Once the first anchor 30a is inserted into the first tissue side 4, the shaped cannula 12 is extended to the second tissue side 6 for insertion of the second anchoring structure 30b to the second tissue side 6. Tension is applied along the threaded member 40 to reducing or limit any excess suture slack as the shaped cannula 12 is rotated and extended towards the second tissue side 6.

In some cases, excessive surrounding tissue can obscure the visibility or working area near the surgical site. The shaped edge 14, associated with the egg-shaped cannula 12, towards the surrounding tissue, can be helpful in separating, displacing, bifurcating or cutting the obscuring tissue. Rotating the cannula tip 16 from the first side 4 towards the second side 6 extends the threaded member 40 from the first side 4 to the second side 6. The second anchoring structure 30b is inserted into the second side 6 after positioning the cannula tip 16 in proximity to the second side 6. After the second anchoring structure 30b is inserted through the second tissue side 6, securing the threaded member 40 to both the first and second tissue sections 4, 6.

The shaped cannula 12 generally extends from a proximate end to a distal end and has a generally elliptical or egg-shaped cross-section with the shaped edge 14 associated with the egg-tip opposite the generally rounded base-region of the cannula 12. The shaped edge 14 is adapted for separating surrounding fascia, thoracic wall or thick muscular tissue, etc. while moving the distal tip 16 between the first side 4 and the second side 6. The distal tip, also referred to herein as tip 16 of the cannula 12 is generally adapted for suturing the defect 2 with the suture assembly 34 by creating tension in the threaded member 40 while inserting anchors 30a through the surrounding tissue.

After anchoring of the threaded member 40 to the second side, the shaped edge 14 (or another shaped surface) may be used to separate the threaded member 40. Each end of the threaded member 40 is secured to one of the anchors 30a, 30b. Generally, the anchor 30 is shaped for piercing the tissue while preventing resistance against premature separation of the surrounding tissue.

It is to be understood that while certain forms of the present invention have been illustrated and described herein, it is not to be limited to the specific forms or arrangement of parts described and shown.

1. An interventional radiology suture device comprising:
a head configured for slidable receipt of a plunger assembly;
a shaped cannula extending outwardly from said head for transmitting a threaded suture;
a central body extending between said head and said shaped cannula,
said central body presenting a channel for receiving a cartridge of threaded sutures; and
said cartridge being in communication with said plunger assembly for dispensing at least one of said threaded suture through said shaped cannula.

2. The interventional radiology suture device according to claim 1 further comprising a radiologically visible tip for dispensing said threaded suture.

3. The interventional radiology suture device according to claim 1 wherein said threaded suture further comprising a first anchor separated from a second anchor by a threaded member.

4. The interventional radiology suture device according to claim 1 wherein said shaped cannula includes a longitudinally extending edge.

5. The interventional radiology suture device according to claim 1 wherein said central body presents a secondary cannula for transmission of a contrasting fluid through said shaped cannula.
